(19) United States
(12) Patent Application Publication
(10) Pub. No.: US 2023/0201505 A1
(43) Pub. Date: Jun. 29, 2023

(54) VENTILATION MONITORING METHOD AND SYSTEM THEREFOR

(71) Applicant: NORTH-WEST UNIVERSITY, Potchefstroom (ZA)

(72) Inventors: Magdalena Johanna GROBLER, Potchefstroom (ZA); Henri-Jean MARAIS, Potchefstroom (ZA); Bas Derk VAN KAMPEN, Riebeeck Kasteel (ZA)

(73) Assignee: NORTH-WEST UNIVERSITY, Potchefstroom (ZA)

(21) Appl. No.: 17/923,742

(22) PCT Filed: May 5, 2021
(86) PCT No.: PCT/IB2021/053792

§ 371 Date: Nov. 7, 2022

(30) Foreign Application Priority Data
May 5, 2020 (ZA) 2020/02432

(51) Int. Cl. A61M 16/00; G16H 40/67

(52) U.S. Cl. CPC A61M 16/026; G16H 40/67; A61M 16/0051; A61M 2205/583; A61M 2205/3553; A61M 2205/84; A61M 2016/0027

(57) ABSTRACT

The invention relates to a ventilation monitoring system comprising a ventilation data capturing device connected between a patient and a ventilator device dedicated to the patient, for collecting ventilation data between the patient and ventilator device; an on-site device; a remote device for communicating with the ventilation data capturing device and the on-site device; at least one processor; and at least one memory device coupled to the at least processor configured to: collect ventilation data from the ventilation data capturing device; transmit the collected ventilation data to the remote device/server for analysis; and generate output data comprising the instructions/commands for actioning on the ventilator device, to provide suitable ventilation support to the patient, or generate output data comprising the status of the ventilator device associated with the ventilation data capturing device.

### FIELD OF INVENTION

THIS invention relates to ventilation monitoring systems and methods, for example, to ventilation monitoring systems and methods which monitor patients receiving artificial ventilation via ventilators.

### BACKGROUND OF INVENTION

Ventilators are mechanical, pneumatic and/or electrically actuated devices which provide respiratory support to people in need of artificial ventilation or respiration by creating positive and negative air pressure gradients between airways and lungs of people in a reciprocal fashion. In this regard, a ventilator is typically connected in flow connection to a suitable arrangement which, depending on whether the ventilation is invasive or non-invasive may comprise a endotracheal tube (ET), mask, ventilator hood/helmet, or the like which is attachable to a person to artificially provide breathable air and/or oxygen to lungs of the person and remove oxygen depleted air therefrom in a reciprocal fashion.

Conventional ventilators are usually electronic and provide ventilation data associated with the ventilation of a person in a processed and understandable format which enables alarms, etc. to be programmed/set whereas older mechanical/pneumatic ventilators provide ventilation data visually by way of suitable gauges, dials, etc. and rely on constant monitoring by skilled healthcare workers.

Notwithstanding, due to the complexity of correctly artificially ventilating a person and complications which could arise with artificially ventilating a person, ventilation, particularly ventilation requiring intubation, is done at a medical care facility such as a hospital. In this way, a patient receiving artificial ventilation is able to be under close medical supervision from a suitable healthcare worker such as a doctor, nurse, or more particularly an Intensive Care Unit (ICU) intensivist.

However, in times of epidemics and pandemics, particularly those associated with viruses which cause respiratory illness, resources are stretched as hospitals may be flooded with ailing patients in need of medical assistance including ventilation. In particular, hospitals may be understaffed with suitable healthcare workers to cater for the surging numbers of patients who may require ventilation support, or, if adequately staffed may not have ample medical staff that are experienced enough to make sound judgements on the type of ventilation support needed by the patients.

In addition to the above scenario, hospitals may occasionally be understaffed when treating patients suffering from head injury; stroke; lung disease; spinal cord injury; polio; sudden cardiac arrest; neonatal respiratory distress syndrome; acute respiratory distress syndrome (ARDS); pneumonia; sepsis; and chronic obstructive pulmonary disease (COPD), which patients may require ventilators from time to time.

In addition, hospitals typically do not have a uniform fleet of ventilators available at the same time, which would make it difficult for inexperienced medical staff to monitor patients based on varying ventilation data formats or types from different ventilators. This problem is exacerbated when conventional sophisticated electronic/electrical ventilators are not available due to shortages in supply and the only alternative available are archaic ventilators of the mechanical/pneumatic type as described above as many healthcare workers would not know how to read the ventilation data from these older ventilators.

In addition, in situations where a patient is homebound, possibly because the hospitals do not have enough beds to accommodate additional patients, there is generally no existing mechanism that can enable a medical practitioner or intensivist to monitor the breathing status of the patient and also monitor the ventilator or breathing devices used by the homebound patient.

The present invention seeks to address at least the abovementioned problems.

### SUMMARY OF INVENTION

According to a first aspect of the invention there is provided a ventilation monitoring system for use in monitoring ventilation of at least one patient being ventilated by a suitable ventilator device via an associated connection arrangement defining a flow path of gas including air or oxygen between the patient and the ventilator device, wherein the system comprises:

a ventilation data capturing device connected between a patient and a ventilator device dedicated to and/or associated with the patient, wherein the ventilation data capturing device is arranged to collect ventilation data from at least the flow path between the patient and the ventilator device;

at least one processor; and

at least one memory device coupled to the at least one processor, wherein the at least one processor is configured to:
collect or receive ventilation data from the ventilation data capturing device; and
generate output data, based on the collected or received ventilation data, wherein the output data comprises or is indicative of a status of the ventilator device, or
generate output data, based on the collected or received ventilation data, comprising at least instructions/commands for actioning on the ventilator device.
   According to a second aspect of the invention there is provided a ventilation monitoring method for monitoring ventilation of at least one patient being ventilated by a suitable ventilator device via an associated connection arrangement defining a flow path of gas including air or oxygen between the patient and the ventilator device, wherein the method comprises:

fitting a ventilation data capturing device between a patient and a ventilator device dedicated to and/or associated with the patient, the ventilation data capturing device being arranged to collect ventilation data from at least the flow path between the patient and ventilator device;

collecting or receiving, by means of at least one processor, ventilation data from the ventilation data capturing device; and

generating, by means of the at least one processor, output data comprising a status of the ventilator device, or

generating, by means of the at least one processor, output data comprising at least instructions/commands for actioning on the ventilator device.

According to a third aspect of the invention there is provided a ventilation monitoring system for use in monitoring ventilation of at least one patient being ventilated by a suitable ventilator device via an associated connection arrangement defining a flow path of air to and from the patient, wherein the system comprises:

a ventilation data capturing device connected between a patient and a ventilator device dedicated to and/or associated with the patient, wherein the ventilation data capturing device is arranged to collect ventilation data between the patient and ventilator device;

an on-site device for use by an on-site medical practitioner;

a remote device for use by a remote medical practitioner for communicating with the ventilation data capturing device and the on-site device;

at least one processor; and

at least one memory device coupled to the at least processor, wherein the processor is configured to:
collect ventilation data from the ventilation data capturing device;
transmit the collected ventilation data to the remote device for analysis by the remote medical practitioner;
collect commands/instructions, from the remote device, for actioning by the on-site medical practitioner on the ventilator device; and
generate output data for output on the on-site device, wherein the output data comprises the instructions/commands for actioning on the ventilator device associated with the ventilation data capturing device by the on-site medical practitioner, to provide suitable ventilation support to the patient.

The at least one processor may be configured to transmit the output data to the on-site device.

The at least one memory device may store non-transitory computer executable instructions which, when executed by the at least one processor, causes the at least one processor to perform functionality described herein.

The connector arrangement may comprise suitable tube/s; a patient connector end; and a ventilator device connector end. The patient connector end may terminate in an endotracheal tube, a mask, a non-invasive hood/helmet. In this regard, it will be understood that the ventilation data capturing device may be attached in flow communication in the connector arrangement between the ventilator device and the patient.

The may be a plurality of ventilator devices and plurality of patients. Each ventilator device may be dedicated to and/or associated with and/or associated with a particular patient. The system may therefore comprise a plurality of ventilation data capturing devices, wherein each ventilation data capturing device of the plurality of ventilation data capturing devices is connected between a dedicated ventilator device and the particular patient. Accordingly, ventilation data collected from each ventilation data capturing device may be transmitted to the remote device for analysis by the remote practitioner. Similarly, the commands collected from the remote device may be for each ventilator device that is associated with the ventilation data capturing device.

Each ventilator device and/or ventilation data capturing device may be uniquely identifiable. In one example embodiment each ventilator device and/or ventilation data capturing device, and/or patient may have a unique identifier associated therewith. In this regard, the at least one processor may be configured to store and associate, in at least one memory device, unique identifiers of at least two of an associated ventilator device, ventilation data capturing device, and patient under a particular ventilation profile or ventilation file. The unique identifier in the case of the patient may be their name. The unique identifier for the devices may be a numeric, character based, or alphanumeric code, a graphical symbol, or the like.

The profile or file may in any event have details of the location of the associated ventilator device, ventilation data capturing device, or patient so that on-site medical practitioners are able to locate a patient requiring any intervention.

It will be appreciated that the plurality of ventilator devices may be uniform, non-uniform in design and functionality, or may be a combination of ventilators which are uniform and non-uniform in design and functionality.

In an embodiment, the ventilation data may include at least pressure data indicative of and/or associated with a ventilation process between the patient and the ventilator device. The ventilation process may be ventilation of the patient in a conventional fashion via the ventilator device connected in flow communication with the patient. In particular, the creation and/or generation of positive and/or negative pressure differentials between the airways and lungs of a patient to support and/or provide for inhalation and/or exhalation of the patient. The pressure data may therefore be indicative of air pressure in the flow path between ventilator device and the patient.

The pressure data may therefore comprise pressure signals, or data indicative thereof, associated with the ventilation process. To this end, the ventilation data capturing device may comprise one or more suitable sensors for sensing air pressure associated with or indicative of the ventilation process and generating pressure signals in response to said sensing. It will be understood that the ventilation data may be indicative of a ventilation condition of the patient.

The ventilation data capturing device may comprise one or more suitable pressure sensors to measure pressure. The ventilation data capturing device may comprise a memory, a communication module and a controller, wherein the controller is configured to transmit ventilation data collected by the device via the one or more sensors to the at least one processor, via the communication module.

In an embodiment, the at least one processor may be configured to:
analyse the ventilation data collected by the ventilation data capturing device; and
generate output data, wherein the output data comprises at least visual representations of the analysed and/or collected data.

It will be appreciated that the above steps may precede the step of transmitting the ventilation data to the remote device.

The output data may comprise diagrams and/or graphs indicative of the ventilation process. For example, pressure v time diagrams; flow v time diagram; and/or air volume v time diagrams.

In an embodiment, the analysis of the ventilation data may include performing process diagnostics on the ventilation data to extract set points of the ventilation process. In other words, the processor may be configured to analyse the ventilation data by determining set points in the ventilation data, wherein the set points are selected from a group comprising an inspiration:expiration ratio; peak inhalation pressure; exhalation pressure; and tidal volume.

The at least one processor may be configured to:
collect, from the on-site device, patient metadata; and
transmit the patient metadata to the remote device for analysis by the remote medical practitioner.
The patient metadata may include information indicative of one or more of the patient's gender, patient's age, ventilation device details, and details of the patient circuit tubing selection (typically standardised at 22 mm but maybe a non-standard tube/hose).

In an embodiment, at least one processor may be configured to:
analyse the patient metadata; and
generate output data, wherein the output data comprises at least visual representations of the analysed metadata.

The processor may be configured to process the above steps prior to the step of transmitting the patient metadata to the remote device.

The visual representations may typically be in a form of graphs.

In an embodiment, the visually represented graphs may also include the collected ventilation data and patient metadata.

The system may comprise an on-site application or module operating on the on-site device. In this way, the on-site application or module is able to communicate with the at least one processor remotely.

Similarly, the system may comprise a remote device application or module operating on the remote device. In this way, the remote application or module is able to communicate with the at least one processor remotely.

The application or module may be a computer program storing a set of non-transitory computer executable instructions, which when executed by a suitable computing device causes said device to function in a manner as described herein.

According to a fourth aspect of the invention there is provided a ventilation monitoring method for monitoring ventilation of at least one patient being ventilated by a suitable ventilator device via an associated connection arrangement defining a flow path of air to and from the patient, wherein the method comprises:

fitting a ventilation data capturing device between a patient and a ventilator device dedicated to and/or associated with the patient, the ventilation data capturing device being arranged to collect at least one ventilation data between the patient and ventilator device;

providing an on-site device for use by an on-site medical practitioner;

providing a remote device for use by a remote medical practitioner for communicating with the ventilation data capturing device and the on-site device;

collecting ventilation data from the ventilation data capturing device;

transmitting the collected ventilation data to the remote device for analysis by the remote medical practitioner;

collecting commands/instructions, from the remote device, for actioning by the on-site medical practitioner on the ventilator device; and

generating output data for output on the on-site device, wherein the output data comprises the instructions/commands for actioning on the ventilator device associated with the ventilation data capturing device by the on-site medical practitioner, to provide suitable ventilation support to the patient.

The method may comprise transmitting the output data to the on-site device.

The method may comprise the steps of:
analysing the ventilation data collected by the ventilation data capturing device; and
generating output data, wherein the output data comprises at least visual representations of the analysed and/or collected data.

It will be appreciated that the above steps may precede transmitting the ventilation data to the remote device.

In an embodiment, the analysing step may comprise performing process diagnostics on the ventilation data to extract set points of the ventilation process. In other words, the method may comprise analysing the ventilation data by determining set points in the ventilation data, wherein the set points are selected from a group comprising an inspiration:expiration ratio; peak inhalation pressure; exhalation pressure; and tidal volume.

The method may further comprise:
collecting, from the on-site device, patient metadata; and
transmitting the patient metadata to the remote device for analysis by the remote medical practitioner.

In an embodiment, the method may comprise:
analysing the patient metadata; and
generating output data, wherein the output data comprises at least visual representations of the analysed metadata.

The visual representations may typically be in a form of graphs.

In an embodiment, the visually represented graphs may also include the collected ventilation data and patient metadata.

According to a fifth aspect of the invention, there is provided a computer-readable medium storing instructions thereon which are executable by at least one processor of a ventilator monitoring system, the ventilator monitoring system comprising:

a ventilation data capturing device connected between a patient and a ventilator device dedicated to and/or associated with the patient, for providing ventilation support to the patient, the ventilation data capturing device being arranged to collect at least one ventilation data between the patient and ventilator device;

an on-site device for use by an on-site medical practitioner; and

a remote device for use by a remote medical practitioner for communicating with the ventilation data capturing device and the on-site device,

wherein the instructions when executed by the at least one processor is arranged to cause the at least one processor to perform the operations of:

collecting ventilation data from the ventilation data capturing device;

transmitting the collected ventilation data to the remote device for analysis by the remote medical practitioner;

collecting commands/instructions, from the remote device, for actioning by the on-site medical practitioner on the ventilator device; and

generating output data for output on the on-site device, wherein the output data comprises the instructions/commands for actioning on the ventilator device associated with the ventilation data capturing device by the on-site medical practitioner, to provide suitable ventilation support to the patient.

According to a sixth aspect of the invention, there is provided a ventilation monitoring system for monitoring ventilation of at least one patient being ventilated by a suitable ventilator device via an associated connection arrangement defining a flow path of air to and from the patient, wherein the system comprises:

at least one processor; and

at least one memory device coupled to the at least processor, wherein the at least one processor is configured to:
collect ventilation data from a ventilation data capturing device connected between a patient and a ventilator device dedicated to and/or associated with the patient;
transmit the collected ventilation data to a remote device for analysis by a remote medical practitioner;
collect, from the remote device, commands/instructions for actioning by an on-site medical practitioner on the ventilator device; and
generate output data for output on the on-site device, wherein the output data comprises at least the instructions/commands for actioning on the ventilator device associated with the ventilation data capturing device by the on-site medical practitioner, to provide suitable ventilation support to the patient.

The system may further comprise a plurality of ventilation data capturing devices, wherein each ventilation data capturing device is connected between a patient and a ventilator device dedicated to and/or associated with the patient.

The system may comprise an on-site application or module operating on an on-site device for use by an on-site medical practitioner. The system may comprise the on-site device with the aforementioned module or application operating thereon.

To this end, the on-site device may be a conventional computing device such as a computer, laptop, or the like

The system may comprise a remote application or module operating on a remote device for use by a remote medical practitioner for communicating with the ventilation data capturing device and the on-site device. The system may comprise the remote device with the aforementioned module or application operating thereon.

To this end, the remote device may be a conventional computing device such as a computer, laptop, or the like, or a cloud computing device.

The at least one processor may be configured to transmit the output data to the on-site device.

The at least one processor may be configured to:
analyse the ventilation data collected by the ventilation data capturing device; and
generate output data, wherein the output data comprises at least visual representations of the analysed and/or collected data.

The output data may comprise diagrams and/or graphs indicative of the ventilation process. For example, pressure v time diagrams; flow v time diagram; and/or air volume v time diagrams.

In an embodiment, the analysis of the ventilation data may include performing process diagnostics on the ventilation data to extract set points of the ventilation process. In other words, the processor may be configured to analyse the ventilation data by determining set points in the ventilation data, wherein the set points are selected from a group comprising an inspiration:expiration ratio; peak inhalation pressure; exhalation pressure; and tidal volume.

The at least one processor may be configured to:
collect, from the on-site device, patient metadata; and
transmit the patient metadata to the remote device for analysis by the remote medical practitioner.

In an embodiment, at least one processor may be configured to:
analyse the patient metadata; and
generate output data, wherein the output data comprises at least visual representations of the analysed metadata.

The visual representations may typically be in a form of graphs.

In an embodiment, the visually represented graphs may also include the collected ventilation data and patient metadata.

According to a seventh aspect of the invention there is provided a ventilation monitoring method for monitoring ventilation of at least one patient being ventilated by a suitable ventilator device via an associated connection arrangement defining a flow path of air to and from the patient, wherein the method comprises:

collecting, by means of at least one processor, ventilation data from a ventilation data capturing device connected between a patient and a ventilator device dedicated to and/or associated with the patient;

transmitting, by means of the at least one processor, the collected ventilation data to a remote device for analysis by a remote medical practitioner;

collecting, by means of the at least one processor, from the remote device, commands/instructions for actioning by an on-site medical practitioner on the ventilator device; and

generating output data for output on the on-site device, wherein the output data comprises the instructions/commands for actioning on the ventilator device associated with the ventilation data capturing device by the on-site medical practitioner, to provide suitable ventilation support to the patient.

The method may comprise transmitting the output data to the on-site device.

The method may comprise fitting a ventilation data capturing device between a patient and a ventilator device dedicated to and/or associated with the patient, the ventilation data capturing device being arranged to collect at least one ventilation data between the patient and ventilator device.

The method may comprise providing an on-site device for use by an on-site medical practitioner. The method may comprise providing an on-site module or application on the on-site device.

The method may comprise providing a remote device for use by a remote medical practitioner for communicating with the ventilation data capturing device and the on-site device. The method may comprise providing a remote module or application on the on-site device.

The method may comprise the steps of:
analysing the ventilation data collected by the ventilation data capturing device; and
generating output data, wherein the output data comprises at least visual representations of the analysed and/or collected data.

It will be appreciated that the above steps may precede transmitting the ventilation data to the remote device.

In an embodiment, the analysing step may comprise performing process diagnostics on the ventilation data to extract set points of the ventilation process. In other words, the method may comprise analysing the ventilation data by determining set points in the ventilation data, wherein the set points are selected from a group comprising an inspiration:expiration ratio; peak inhalation pressure; exhalation pressure; and tidal volume.

The method may further comprise:
collecting, from the on-site device, patient metadata; and
transmitting the patient metadata to the remote device for analysis by the remote medical practitioner.

In an embodiment, the method may comprise:
analysing the patient metadata; and
generating output data, wherein the output data comprises at least visual representations of the analysed metadata.

The visual representations may typically be in a form of graphs.

In an embodiment, the visually represented graphs may also include the collected ventilation data and patient metadata.

According to an eighth aspect of the invention, there is provided a computer-readable medium storing instructions thereon, which when executed by at least one processor is arranged to cause the at least one processor to perform the operations of:

collecting, by means of at least one processor, ventilation data from a ventilation data capturing device connected between a patient and a ventilator device dedicated to and/or associated with the patient;

transmitting, by means of the at least one processor, the collected ventilation data to a remote device for analysis by a remote medical practitioner;

collecting, by means of the at least one processor, from the remote device, commands/instructions for actioning by an on-site medical practitioner on the ventilator device; and

generating output data for output on the on-site device, wherein the output data comprises the instructions/commands for actioning on the ventilator device associated with the ventilation data capturing device by the on-site medical practitioner, to provide suitable ventilation support to the patient.

In an embodiment, the step of transmitting the ventilation data to the remote device may be preceded by the steps of:
analysing the ventilation data collected by the ventilation data capturing device; and
generating output data, wherein the output data comprises at least visual representations of the analysed and/or collected data.

In an embodiment, the analysing of the ventilation data may include performing, by means of the at least one processor, process diagnostics on the ventilation data to extract set points of the ventilation process.

In an embodiment, the step of transmitting the ventilation data to the remote device may optionally be preceded by, or followed by, the steps of:
collecting, from the on-site device, by means of the at least one processor, patient metadata; and
transmitting, by means of the at least one processor, the patient metadata to the remote device for analysis by the remote medical practitioner.

In an embodiment, the step of transmitting the patient metadata to the remote device may be preceded by the steps of:
analysing the patient metadata; and
generating output data, wherein the output data comprises at least visual representations of the analysed metadata.

In an embodiment, the graphs may also include the collected ventilation data and patient metadata.

According to a ninth aspect of the invention there is provided a ventilation device comprising the ventilation data capturing device as hereinbefore described.

According to a tenth aspect of the invention there is provided a method of modifying an existing ventilator device, the method comprising fitting the ventilation data capturing device as hereinbefore described to a ventilator device.

According to an eleventh aspect of the invention there is provided a ventilator kit comprising a ventilator device; and a ventilation data capturing device as hereinbefore described.

It will be understood that descriptions directed to one aspect of the invention described herein may be applicable mutatis mutandis to other aspects of the invention. Moreover, the numbering of the various aspects of the invention do not in any way constitute a ranking of said aspects of the invention.

It will also be appreciated that, in the context of the invention, the words ventilator device and ventilator are used exchangeably. In addition, the definition of ventilator device in the context of the invention extends to breathing aid devices in addition to the conventional machines used in ICU wards, but then also less complex systems providing breathing support and oxygen therapy, including oxygen canisters in a home care setting.

### BRIEF DESCRIPTION OF DRAWINGS

The objects of this invention and the manner of obtaining them, will become more apparent, and the invention itself will be better understood, by reference to the following description of embodiments of the invention taken in conjunction with the accompanying diagrammatic drawings, wherein:

FIG. 1 shows a network of an example embodiment of a ventilation monitoring system in accordance with an example embodiment of the invention;

FIG. 2 shows a process flow block diagram of an operational flow of a ventilation monitoring system and/or method in accordance with an example embodiment of the invention;

FIG. 3 shows a process flow block diagram view of an operational flow of the setup of a ventilation monitoring system in accordance with an example embodiment of the invention;

FIG. 4 shows a process flow block diagram of an operational flow of a Standard Operating Procedure (SOP) of the medical staff;

FIG. 5 shows a process flow block diagram of an operational flow of a calibration stage of a ventilation monitoring method and/or system in accordance with an example embodiment of the invention;

FIG. 6 shows a process flow block diagram of a functional flow of a ventilation monitoring system and/or method in accordance with an example embodiment of the invention;

FIG. 7 shows schematic drawing of an architecture of an inline ventilation monitoring device of a ventilation monitoring system in accordance with an example embodiment of the invention;

FIG. 7B shows a ventilation data capturing device of the ventilation monitoring system in accordance with the invention, being fitted to a tube a of ventilator device;

FIG. 8 shows a process flow block diagram of a data capturing algorithm of a ventilation monitoring system and/or method in accordance with the invention;

FIG. 9 shows a process flow block diagram of an automatic process diagnostic algorithm of a ventilation monitoring system and/or method in accordance with an example embodiment of the invention;

FIG. 10 shows a pictorial view of typical pressure curves associated with a ventilation process of a ventilation monitoring system and/or method in accordance with an example embodiment of the invention;

FIG. 11 shows a schematic diagram of an architecture of a web-based monitoring dashboard of a ventilation monitoring system in accordance with an example embodiment of the invention;

FIG. 12 shows a high-level block flow diagram of a ventilation monitoring method in accordance with an example embodiment of the invention; and

FIG. 13 shows a diagrammatic representation of a machine in the example form of a computer system in which a set of instructions for causing the machine to perform any one or more of the methodologies discussed herein, may be executed.

### DETAILED DESCRIPTION OF AN EXAMPLE EMBODIMENT

The following description of the invention is provided as an enabling teaching of the invention. Those skilled in the relevant art will recognise that many changes can be made to the embodiment described, while still attaining the beneficial results of the present invention. It will also be apparent that some of the desired benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention. Thus, the following description is provided as illustrative of the principles of the present invention and not a limitation thereof.

It will be appreciated that the phrase “for example,” “such as”, and variants thereof describe non-limiting embodiments of the presently disclosed subject matter. Reference in the specification to “one example embodiment”, “another example embodiment”, “some example embodiment”, or variants thereof means that a particular feature, structure or characteristic described in connection with the embodiment(s) is included in at least one embodiment of the presently disclosed subject matter. Thus, the use of the phrase “one example embodiment”, “another example embodiment”, “some example embodiment”, or variants thereof does not necessarily refer to the same embodiment(s).

Unless otherwise stated, some features of the subject matter described herein, which are, described in the context of separate embodiments for purposes of clarity, may also be provided in combination in a single embodiment. Similarly, various features of the subject matter disclosed herein which are described in the context of a single embodiment may also be provided separately or in any suitable sub-combination.

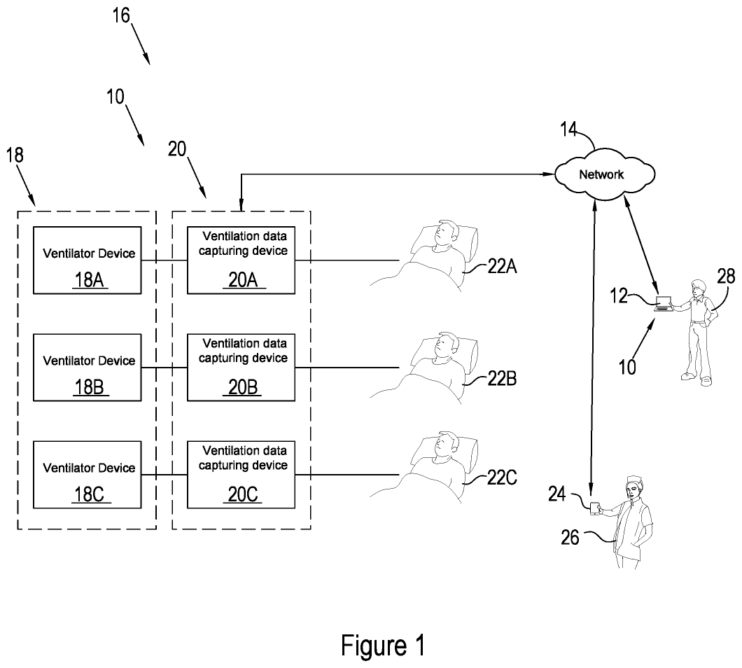

Referring to FIG. 1 of the drawings, there is provided a network 16 incorporating a ventilation monitoring system 10 in accordance with an example embodiment of the invention. The ventilation monitoring system 10 is typically deployed to facilitate at least remote monitoring of ventilation data associated with the ventilation of a plurality of people, for example, in a hospital. However, nothing precludes system 10 described herein from having application where the plurality of people being ventilated are geographically separated, for example where some of the patients are homebound and others are located in a hospital or other facilities.

In the shown embodiment of the invention, the ventilation monitoring system 10 comprises a plurality of isolated, ventilation data capturing devices 20A, 20B, 20C each of which being respectively connected, in an in-line flow communicative fashion in the flow path between ventilator device 18A, 18B, 18C and a patient 22A, 22B, 22C. As mentioned before, some of the patients may be in the same building such as a hospital, while others may be homebound and located remotely from the hospital.

The ventilation monitoring system 10 further comprises a remote device 12 with a suitable remote module or application operating thereon. The application or module may be a software application or module and in one example embodiment may be in the form of a web-based monitoring dashboard 12 for use by a remote medical practitioner, typically a human intensivist 28, as will be described in more detail below. The network 16 further comprises an onsite device 24 with a suitable on-site module or application operating thereon. The application or module may be a software application or module and may be used by an on-site medical practitioner 26, as will be described below, in a setting where the patients are located in the hospital or same building. In a situation where one or more patients are located remotely from the hospital, for example, situated at their own homes, the on-site device may be a device running suitable software in accordance with the invention, and said on-site device may be associated with a person taking care of the patient and/or may be associated with the patient taking care of him/herself.

The on-site device 24, the ventilation data capturing devices 20A, 20B, 20C and the remote device 12 are in communication at least with one another by a communication network 14.

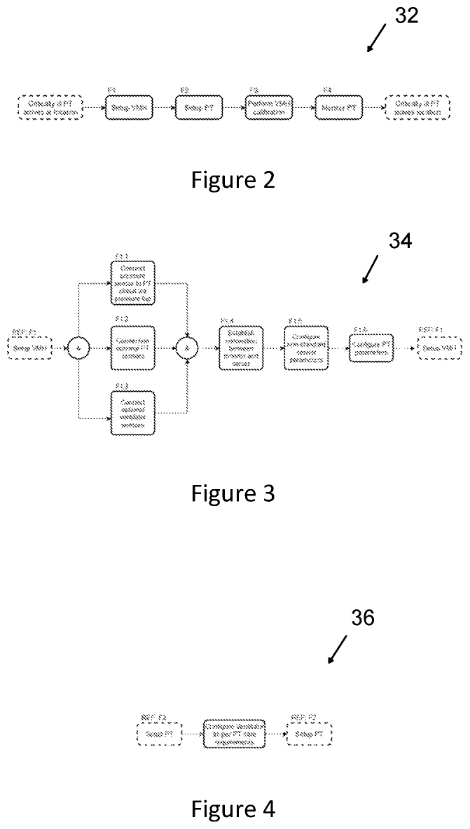

FIG. 2 shows the overall flow of the ventilation process 32 from the moment a critically ill patient reports for ventilation until the patient no longer requires ventilation. In the FIGS. 2 to 6, the various sub processes denoted by F1 to F4 in FIG. 2 will be described in more detail. This includes the setup of the Ventilation Monitoring Hardware F1 (i.e. set up of the ventilation data capturing device), Patient (PT) setup F2 (as is known in the art), calibration of the VMH F3, and the patient monitoring phase F4 (as is known in the art).

FIG. 3 shows a process view of the operational flow of the setup of the ventilator monitoring hardware (i.e. the ventilation data capturing device) 34. The VMH setup phase F1 is described in FIG. 3 entails the correct connection of all the relevant sensors between the ventilator and the patient F1.1-F1.3, after which a connection between the monitor and the server should be established in F1.4. Thereafter non-standard sensor parameters F1.5 and patient parameters F1.6 are configured. FIG. 4 shows the basic Standard Operating Procedure (SOP), 36, of the medical staff when configuring the ventilator device 18A, 18B, 18C as is known in the art and the specific steps of configuring the ventilator device are not relevant to the present invention and will thus not be described.

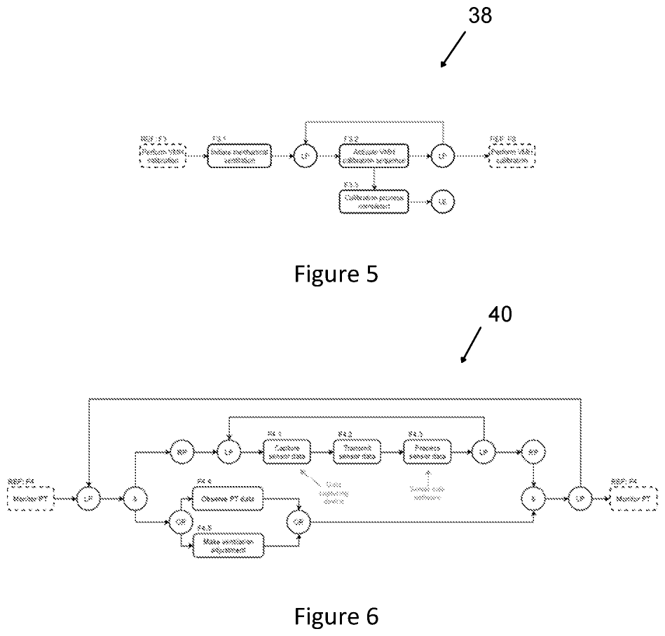

FIG. 5 shows the calibration phase 38 of the ventilator device which entails initiation of mechanical ventilation F3.1, after which a specific calibration sequence F3.2 is performed in a loop until the operating point of the ventilation process stabilises, at which point the calibration process is completed F3.3.

Returning back to FIG. 1, the communications network 14 may comprise one or more different types of communication networks. In this regard, the communication networks may be one or more of the Internet, a local area network (LAN), a wide area network (WAN), a metropolitan area network (MAN), various types of telephone networks (e.g., Public Switch

Telephone Networks (PSTN) with Digital Subscriber Line (DSL) technology) or mobile networks (e.g., Global System Mobile (GSM) communication, General Packet Radio Service (GPRS), Code Division Multiple Access (CDMA), and other suitable mobile telecommunication network technologies), or any combination thereof. It will be noted that communication within the network may achieved via suitable wireless or hard-wired communication technologies and/or standards (e.g., wireless fidelity (Wi-Fi®), 4G, long-term evolution (LTE™), WiMAX, 5G, and the like). In some example embodiments, the system 10 may be coupled to other elements of the communications network 14 via dedicated communication channels, for example, secure communication networks in the form of encrypted communication lines (e.g. SSL (Secure Socket Layer) encryption).

Though not illustrated, it will be understood that the system 10 may include one or more of a back-end (e.g., a data server), a middleware (e.g., an application server), and a front-end (e.g., a client computing device having a graphical user interface (GUI) or a Web browser through which a user can interact with example implementations of the subject matter described herein).

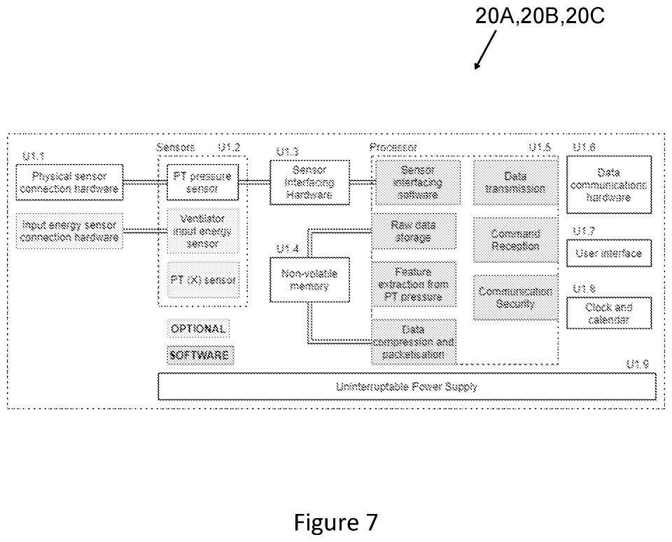

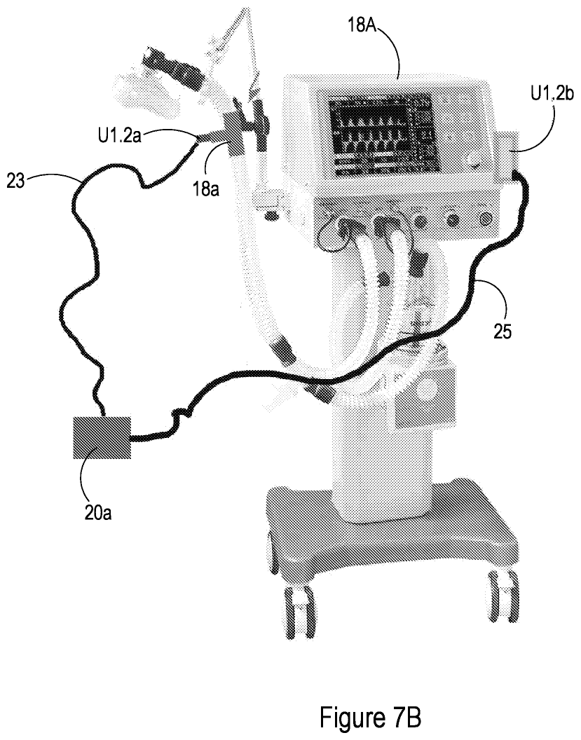

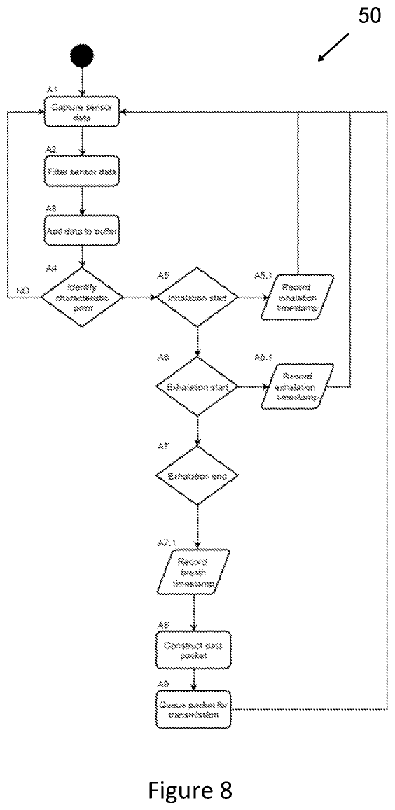

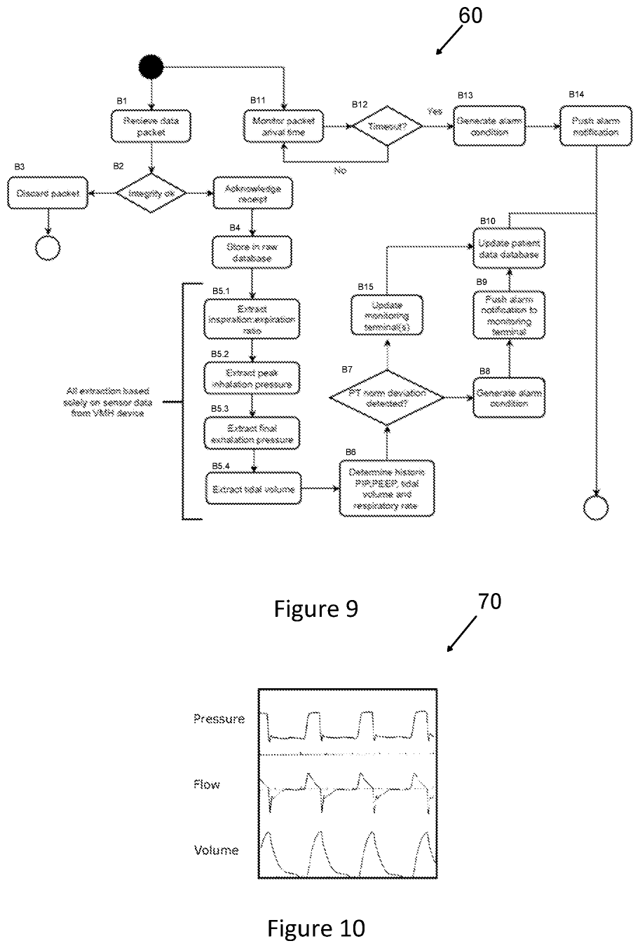

With reference now also to FIG. 7 of the drawings, each ventilation data capturing device 20A, 20B, 20C comprises a physical sensor connection U1.1 and a collection of sensors U1.2 including a patient air pressure sensor for measuring the pressure in the flow path to the patient; a ventilator input energy sensor; and any other optional patient and ventilator sensors, such as % O2, Relative Humidity, and temperature sensors, which can be contemplated. As shown in FIG. 7B, the patient air pressure sensor U1.2a is fitted, typically retrofitted, to a tube 18a of a ventilator device 18, and is connected to the ventilator data capturing device 20A by a suitable cable 23, and the input energy sensor U1.2b that is connected to the ventilation data capturing device 20A by a suitable cable 25 is fitted to the ventilator device 18. For example, in electrically powered ventilators the ventilator input energy sensor would indicate that the ventilator itself has lost power. However, in pneumatic ventilators, the ventilator input energy sensor would be replaced by a pressure sensor arranged to detect lack of input pressure which is synonymous with a “power” failure. The collection of sensors U1.2 are connected via sensor interfacing hardware U1.3 to a processor U1.5 with non-volatile memory U1.4 containing software or computer instructions adapted to cause the processor to perform operations 50, 60 as described in FIGS. 8 and 9 below. Data communication hardware U1.6 is utilised to communicate data to a user interface U1.7. For recordkeeping and trend analysis over time, a clock and calendar, U1.8, are incorporated in each ventilation data capturing device 20A, 20B, 20C. Since stable power supply cannot always be guaranteed and this is a time and connection sensitive monitoring process, an uninterruptable power supply (UPS), U1.9, is also incorporated in each of the ventilation data capturing devices 20A, 20B, 20C.

In another embodiment, for example in a situation whether the patient is homebound and the ventilator device used is a simple oxygen canister connected to an oxygen mask via a suitable tube, and the ventilation capturing device being fitted in-line along the airflow defined by the tube extending between the oxygen mask and oxygen cannister, the ventilation capturing device may optionally not comprise the processor and the memory as described above, but the ventilation capturing devices 20A, 20B, 20C may only be arranged to capture ventilation data between the patient and the ventilator device, and transmit the ventilation data to a remote server which may include the remote device 12 which may comprise at least one processor and at least one memory device containing instructions for performing the operations of the present invention, and which operations can be visually represented on a display screen associated with the server.

In the context of the present invention, it will be understood that ventilation or ventilator device includes the provision of a positive flow of air/oxygen to a particular patient.

As envisaged in the present invention, the ventilation data capturing devices 20A, 20B, 20C and the remote device 12 comprise processors and memory devices which are arranged to perform the operations of the present invention as will be described in detail below when making reference to FIGS. 8 and 9.

Typically, the sensors of each ventilation data capturing device 20A, 20B, 20C are arranged to capture F4.1 the relevant ventilation data between their respective patients 22A, 22B, 22C and the ventilator devices 18A, 18B, 18C, as shown in the process diagram 40 on FIG. 6. The sensor data, including air/oxygen line pressure data (such as the data about air/oxygen pressure flowing to and from the ventilator device to the patient), is transmitted F4.2 and processed F4.3 by the processor U1.5, as shown in FIGS. 7 and 8, and the remote medical practitioner, typically an intensivist 28, can further observe patient data F4.4 on the remote device 12 by means of the operations of the system as shown in FIG. 9, which would then prompt the remote medical practitioner to send instructions or commands or recommendations to the on-site medical staff or person associated with the on-site device (when the patient is homebound or located remotely from the hospital) 24 to make adjustments on the ventilator device associated with the patient F4.5, if needed.

In an embodiment, the pressure data can be used to determine the pressure of the gas (i.e. air/oxygen) from a gas supply source of the ventilator device that is arranged to provide positive gas flow to the patient, and when the gas pressure, as determined either by the in-line ventilation data capturing device 20A, flowing to the patient is below a predetermined set point or when the pressure is substantially higher than normal or a predefined threshold, a suitable alarm may be generated by the system 10 to notify the remote device 12 and/or the on-site device 24 that the gas pressure from the ventilator device 18A is substantially lower or higher than normal to provide suitable/adequate ventilation to the patient.

In an embodiment, the gas pressure data can be used to determine the status or condition of the ventilator device 18A. When there is no power supplied to the ventilator device, the ventilator device will typically cease to function, and a lack of flow of gas or a reduction in the flow of gas from the ventilator device will be detected by the sensors associated with the ventilation data capturing device 20A. The data corresponding to the lack of flow of gas (i.e. oxygen/air) or reduction in the flow of gas between the ventilator device 18A and the patient will be communicated to the remote server 12 for processing. Typically, the system 10 will generate a suitable alarm when there is no flow of gas measured/detected by the sensors of the ventilation data capturing device 20A.

In another embodiment, the status of the ventilator device, for example when the ventilator device comprises a gas cylinder connected to a gas mask wearable by a patient and the ventilation data capturing device is fitted in-line between the gas mask (not shown) and gas cylinder (not shown), may be determined by comparing the original amount of gas available in the gas cylinder that is associated with the ventilation data capturing device with the amount of gas available in the gas cylinder at any point in time. The amount of gas stored in the cylinder at any point in time may be determined by performing calculations that determine, in real-time, the rate of flow of gas from the cylinder to the patient and thereby determine the amount of gas that remains in the gas cylinder. The information about the amount of gas in the gas cylinder and flow rate of the gas travelling from the gas cylinder to the patient is arranged to be measured by one or more sensors and/or a flowmeter of the ventilation data capturing device 20A, and the data is stored in-real time in the database or one or more memory devices of the system 10. When the amount of gas available in the gas cylinder at any point in time falls below a particular threshold, the system 10 is arranged to generate a suitable alarm to notify the intensivist or on-site medical practitioner or caregiver or patient, about the status of the ventilator device.

As mentioned before, it will be appreciated that the remote medical practitioner may be a human. However, it is envisaged that the remote medical practitioner may be in the form of a trained Al engine that is arranged to collect data from the ventilation data capturing device and automatically analyse the data to provide suitable outputs/commands for actioning on the ventilator device and outputs/commands that pertain to the status of the ventilator device 18A.

The processor U1.5 is coupled to the memory device U1.4 (including transitory computer memory and/or non-transitory computer memory), which are configured to perform various data processing and communication operations associated with the system 10 as contemplated herein. Similarly, the processor (not shown) on the remote device/server 12 is coupled to a memory device (not shown).

The processor U1.5 (and that of the remote device) may be one or more processors in the form of programmable processors executing one or more computer programs to perform actions by operating on input data and generating an output. The processor U1.5, as well as any computing device referred to herein, may be any kind of electronic device with data processing capabilities including, by way of non-limiting example, a general processor, a graphics processing unit (GPU), a digital signal processor (DSP), a microcontroller, a field programmable gate array (FPGA), an application specific integrated circuit (ASIC), or any other electronic computing device comprising one or more processors of any kind, or any combination thereof. For brevity, steps described as being performed by the system 10 may be steps which are effectively performed by the processor U1.5 (and/or the processor of the remote server 12) and vice versa unless otherwise indicated.

It will be appreciated that the memory device U1.4 may be in the form of computer-readable medium including system memory and including random access memory (RAM) devices, cache memories, non-volatile or back-up memories such as programmable or flash memories, read-only memories (ROM), etc. In addition, the memory device U1.4 may be considered to include memory storage physically located elsewhere in the system 10, e.g. any cache memory in the processor U1.5 as well as any storage capacity used as a virtual memory, e.g., as stored on a mass storage device.

Though not illustrated, it will be appreciated that the system 10 may comprise one or more user input devices (e.g., a keyboard, a mouse, imaging device, scanner, microphone) and one or more output devices (e.g., a Liquid Crystal Display (LCD) panel—typically to enable the intensivist 28 to observe the processed ventilation data, a sound playback device (speaker), switches, valves, etc.).

It will be appreciated that the computer programs executable by the processor U1.5 (and that of the remote device/server 12) may be written in any form of programming language, including compiled or interpreted languages, declarative or procedural languages, and can be deployed in any form, including as a stand-alone program or as a module, component, subroutine, object, or other unit suitable for use in a computing environment. The computer program may, but need not, correspond to a file in a file system. The program can be stored in a portion of a file that holds other programs or data (e.g., one or more scripts stored in a mark-up language document), in a single file dedicated to the program in question, or in multiple coordinated files (e.g., files that store one or more modules, sub-programs, or portions of code). The computer program can be deployed to be executed by one processor U1.5 or by multiple processors, even those distributed across multiple locations, for example, in different servers and interconnected by the communication network 14.

The computer programs may be stored in the memory device U1.4 (similar, the applicable/relevant computer programs may also be stored in the memory device of the server 12) or in memory provided in the processor U1.5 (or processor of the server). Though not illustrated or discussed herein, it will be appreciated by those skilled in the field of the invention that the system 10 may comprise a plurality of logic components, electronics, driver circuits, peripheral devices, etc., not described herein for brevity.

The memory device U1.4 contains instructions which are arranged to cause the processor U1.5 to perform the operations 50 as shown in FIGS. 8.

As shown in FIG. 8, at Block A1, each ventilation data capturing device 20A, 20B, 20C captures the sensor data in a form that is compliant with the output format of the sensors. Depending on the type and nature of the sensor this might be analogue form or digitally. At least a single pressure sensor data is captured. Subsequently additional sensors' data is captured according to a similar protocol.

At block A2, the data is filtered to remove unwanted frequency components from the input source. In general, at least a low-pass filter is deployed with a cascade of additional filters not being excluded. Such a cascade would allow a selected band of interest to be identified.

At block A3, sufficiently filtered data is added to a data buffer for processing. The buffer is sized such as that at least 3 breath cycles' worth of sensor data can be stored in a form of volatile memory. The purpose of the data buffer is to allow a manner of historic data (local to the ventilation data capturing device 20A, 20B, 20C) that is used during the extraction phase of the algorithm. At block A4, the computer instructions stored in the volatile memory U1.4 are executed by the processor U1.5 to consider the historic contents of the data buffer so as to generate signals based on the identification of characteristic points. The characteristic points are related to the various physiological phases of human breath and includes at least the identification of the start of inhalation, start of exhalation, and completion of exhalation from the (at least) patient circuit pressure data. The computer instructions for the point extraction is based on the mathematical characteristics of a time varying waveform and makes use of elementary calculus and statistics.

Typically, at block A4, a signal will be generated that a characteristic point has been identified, the signal is interpreted at block A5, and if found to be the start of inhalation, the time that this signal was generated is captured from a clock and calendar module U1.8 at block A5.1. The raw data in the buffer (that leads to the identification) and the timestamp (accurate to at least 100 ms) is stored in the non-volatile memory U1.4 local to the ventilation data capturing device 20A, 20B, 20C. Also, at block A4, a signal will be generated that a characteristic point has been identified, the signal is interpreted at block A6, and if found to be the start of exhalation, the time that this signal was generated is captured from the clock and calendar module U1.8 at block A6.1. The raw data in the buffer (that lead to the identification) and the timestamp (accurate to at least 100 ms) is stored in the non-volatile memory U1.4 local to the particular ventilation data capturing device 20A, 20B, 20C.

At block A4, when a signal that is generated signals that a characteristic point has been identified, the signal is interpreted at block A7 and if found to be the start of exhalation, the time that this signal was generated is captured from the clock and calendar module U1.8 at block A7.1. The raw data in the buffer (that lead to the identification) and the timestamp (accurate to at least 100 ms) is stored in non-volatile memory U1.4 local to the ventilation data capturing device 20A, 20B, 20C.

At this point the non-volatile memory contains the filtered sensor data (of at least the PT circuit's pressure data) and the 3 timestamps associated with the critical points. At block A8, the data is assembled into a data packet comprising some header, payload, and footer. The header of the packet will contain certain identifying information (that ensure data provenance) and other protocol specific information. The payload will consist of the filtered data and the relevant time stamps of at least the patient pressure circuit. Other sensor data could be combined into the payload should this be available. The footer of the packet is used to ensure the integrity of the packet and is calculated by the ventilation data capturing device 20A, 20B, 20C on the header and payload combination. The integrity check is stored in the footer section of the packet.

At block A9, the processor U1.5 ensures that the packet is transmitted to the server 12 by means of the relevant communication interface (wired or wireless). Each packet is delivered to the server 12 only once. As such, at block A9, the processor U1.5 employs suitable logic to handle packet enqueueing, dequeuing, and automatic retransmission.

Now turning our attention to FIG. 9, showing a process diagram flow 60 at the server 12 end of the system 10, at block B1, the server 12 provides a mechanism for the reception of packets that abstracts the underlying physical transmission process. Basic packet integrity checks such as checksums are addressed here. Should a packet fail basic redundancy checks it is not received and the server abstraction performs the required actions for rejected packets.

At block B2, the received packet's integrity is checked in terms of transmission details, timestamps, and other relevant metrics that identifies the provenance of the packet. More importantly only packets from the ventilation data capturing device 20A, 20B, 20C that are authenticated by the server are accepted.

At block B3, the packets from authenticated ventilation data capturing devices 20A, 20B, 20C that pass all integrity tests are acknowledged to the sending ventilation data capturing device 20A, 20B, 20C. Packets that fail are rejected without any response to the sender (i.e. the ventilation data capturing device). This ensures that the system is not vulnerable to bad actors or compromised ventilation data capturing device 20A, 20B, 20C overloading the server 12.

At block B4, the packet that was constructed by the ventilation data capturing device 20A, 20B, 20C is unpacked and stored in a sensor database. It is likely that several versions of block B4 could be deployed in parallel to handle server load or variations in sensor data. However, the sensor database is generic and has minimum data requirements (being patient pressure data and timestamps of characteristic points). Additional data is identified and stored in the database without interpretation.

Based on stored data, the ratio of inhalation to exhalation is determined at block B5.1, by a simple algebraic sum. From the sensor data (and the clock and time module U1.8), the inhalation start time and the exhalation start time are known. This information is used to determine the inhalation time or volume (whichever is more relevant). For the exhalation, a similar process is followed by the examination of the difference between the end of exhalation and the start of exhalation. The IER (i.e. inhalation-expiration ratio) is accordingly stored in a patient database, at block B10.

By means of elementary calculus, the peak inspiration pressure (PIP) as well as the timestamp of occurrence is determined at block B5.2, and stored in the patient database, at block B10.

By means of elementary calculus the peak expiratory pressure (PEEP) is determined at block B5.3, is then classified as PEEP, and stored in the patient database , at block B10, together with the timestamp.

Physical relations are used to determine the volume of air exchanged by the patient (known as tidal volume (TV)) at block B5.4. The primary input to this process is the timestamps of the characteristics points together with the pressure data in the patient circuit and the nominal bore of the interconnecting patient circuit. The nominal bore is typically standardised but this can be adjusted as required.

Historic PIP, PEEP, TV, and IER values are extracted from the patient database (i.e. at block B10). Statistical tools are then used to determine the underlying characteristics of the historic PIP, PEEP, TV, and IER values. Based on intensivist requirements and clinical relevance, the window of comparison can be selected.

A comparison, substantially in real-time, at block B7, between the historic PIP, PEEP, TV, and IER values and the current set of PIP, PEEP, TV, and IER values (at block B6) indicates either that the patient is responding as expected (thus behaviour is statistically similar to historic trend) or that the patient is not responding as expected.

Should the patient not be responding as expected an alarm condition is signalled at block B8. It should be noted that this alarm is not dependent on set limits by the intensivist of the medical practitioner. Rather it is solely based on the patient's current response vs historic response. This will allow a significant reduction in intensivist workload but also more granularity in terms of patient specific alarms. Any physiological parameter that is beyond that which is considered medically safe is automatically signalled as an alarm condition regardless of statistical estimations.

Should the expected patient response be within norms, the monitoring interface (i.e. display device associated with the server 12 or the endpoint device of the intensivist that is coupled with the server 12) is simply updated with the latest information, at block B15.

Alarm conditions are pushed, at Block B10, towards the monitoring intensivist. This will be dependent on the technology used and could be a simple textual message (SMS, email, etc.) or a graphical alert on the monitoring interface. Regardless of the technology used, the defining characteristic of a pushed message is that the information is conveyed to the recipient (i.e. intensivist) in a near real-time manner.

The monitoring terminal(s) is updated and the presence of an alarm condition is also indicated herein.

The patient database, at block B10, is updated with the current PIP, PEEP, TV, and IER values for subsequent monitoring cycles.

With respect to blocks B11 to 14, upon arrival of a data packet from an attached ventilation data capturing device 20A, 20B, 20C, a watchdog timer is reset. Based on the interpacket arrival times a typical packet arrival time is determined per ventilation data capturing device 20A, 20B, 20C. In the event that a packet has not been received from a ventilation data capturing device 20A, 20B, 20C within the expected time window and alarm condition is generated (at block B13) and the alarm is pushed to the responsible person (at block B14).

The system 10, typically the server 12, is accordingly arranged to generate pressure curves 70 as shown in FIG. 10, which will be transmitted and/or displayed on the monitoring interface associated with the server 12 (or the endpoint device (not shown)) of the intensivist) for analysis by the intensivist 28.

The on-site device 26 or any computing device (such as the endpoint device which may be used by the intensivist in the version where the server 12 is located remotely from the intensivist 28) contemplated herein, may comprise one or more computer processors and a computer memory (including transitory computer memory and/or non-transitory computer memory), configured to perform various data processing operations. The devices 26 (and the endpoint device mentioned herein) also include a network communication interface (not shown) to connect to the system 10 via the network 14. Examples of the devices represented by the device 26 may be selected from a group comprising a personal computer, portable computer, smartphone, tablet, notepad, dedicated server computer devices, any type of communication device, and/or other suitable computing devices. It will be appreciated that in some example embodiments, the device 26 may be connected to the network 14 via an intranet, an Internet Service Provider (ISP) and the Internet, a cellular network, and/or other suitable network communication technology.

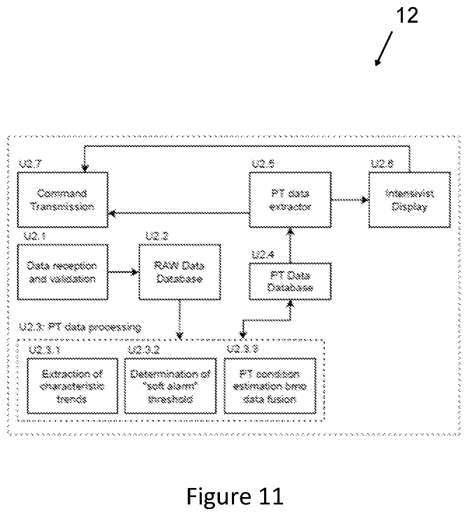

FIG. 11 is a schematic depiction of the software elements, 12, and the applicable interconnections to realise the process operations depicted in FIG. 9. Each of the units of FIG. 11 represents either a custom block of software or off the shelf software components. The units/modules U2.1 and U2.7 are dependent on the data transmission mechanism of the ventilation data capturing device 20A, 20B, 20C and may include commercial software modules or simply be raw TCP/IP datagrams. The modules U2.2 and U2.4 comprises a database (either relational or non-relational) and it is expected that this be off the shelf software modules. The modules U2.3 and U2.5 are software elements that house the various parts of the algorithm described in FIG. 9. Then patient data unit/module U2.3 and its subunits are used to process the data before the processed data is stored in the patient database U2.4.

A patient data extractor U2.5 is then used to provide sensible information to the intensivist 22 via the Intensivist Display U2.6. The display U2.6 is any suitable display (such as the monitoring interface described above) presented to the intensivist and could be a web-page, smartphone application or stand-alone computer application. The defining characteristic of U2.6 is that it provides a means for the intensivist to be a) notified of alarm conditions b) action alarm conditions c) monitor a host of patients simultaneously (preferably at least 1, or 4, and not more than 16) and d) communicate to the local clinician (i.e. the on-site medical practitioner), in the case that the patients are monitored by local clinician in the hospital, or communicate with the patient and/or person caring for the patient in a situation where the patient is located remotely from the hospital, to effect set-point adjustments, etc. on the ventilator device 18A, 18B, 18C. The intensivist 28 can then generate commands to be sent via the command transmission unit U2.7 to the on-site device 26 for analysis by the on-site medical staff 22 or patient and/or person taking care of the patient.

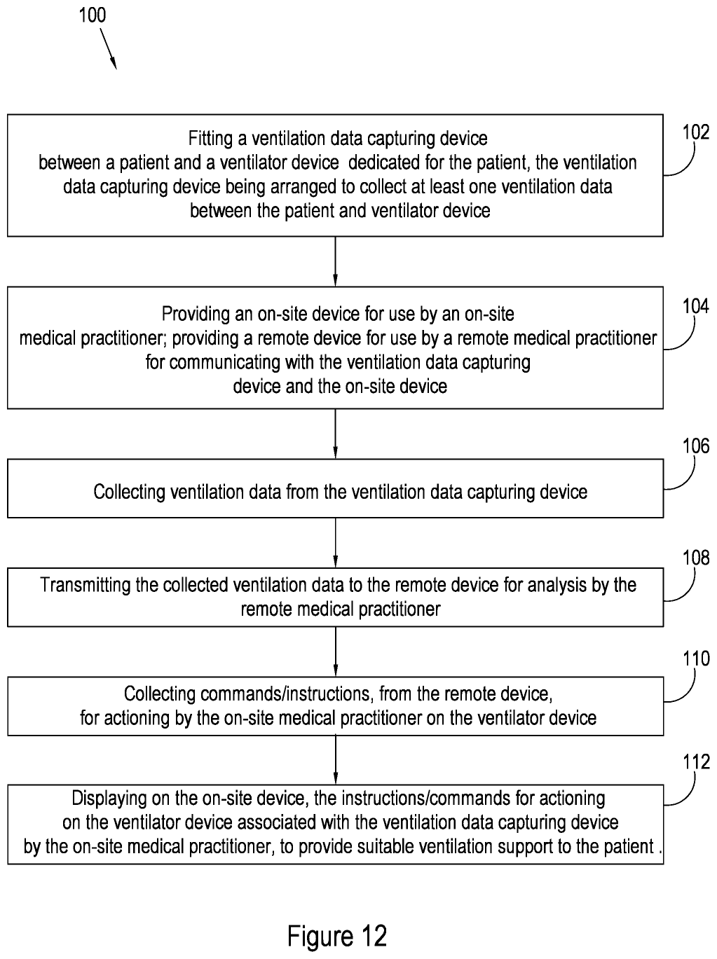

Referring now to FIG. 12 of the drawings where a high-level flow diagram of a method in accordance with an example embodiment of the invention is generally indicated by reference numeral 100. It will be appreciated that the example method 100 may be implemented by systems and means not described herein. However, by way of a non-limiting example, reference will be made to the method 100 as being implemented by way of the system 10, as described above.

Accordingly, the method 100 comprises the steps of: fitting a ventilation data capturing device in-line between a patient and a ventilator device dedicated for the patient 102, the ventilation data capturing device being arranged to collect at least one ventilation data between the patient and ventilator device; providing an on-site device for use by an on-site medical practitioner (or the patient or a person taking care of the patient in a situation where the on-site medical practitioner is unavailable); providing a remote device for use by a remote medical practitioner for communicating with the ventilation data capturing device and the on-site device 104; collecting ventilation data from the ventilation data capturing device 106; transmitting the collected ventilation data to the remote device for analysis by the remote medical practitioner 108; collecting commands/instructions, from the remote device, for actioning by, for example, the on-site medical practitioner on the ventilator device 110; and displaying on the on-site device, the instructions/commands for actioning on the ventilator device associated with the ventilation data capturing device by, for example, the on-site medical practitioner, to provide suitable ventilation support to the patient 112.

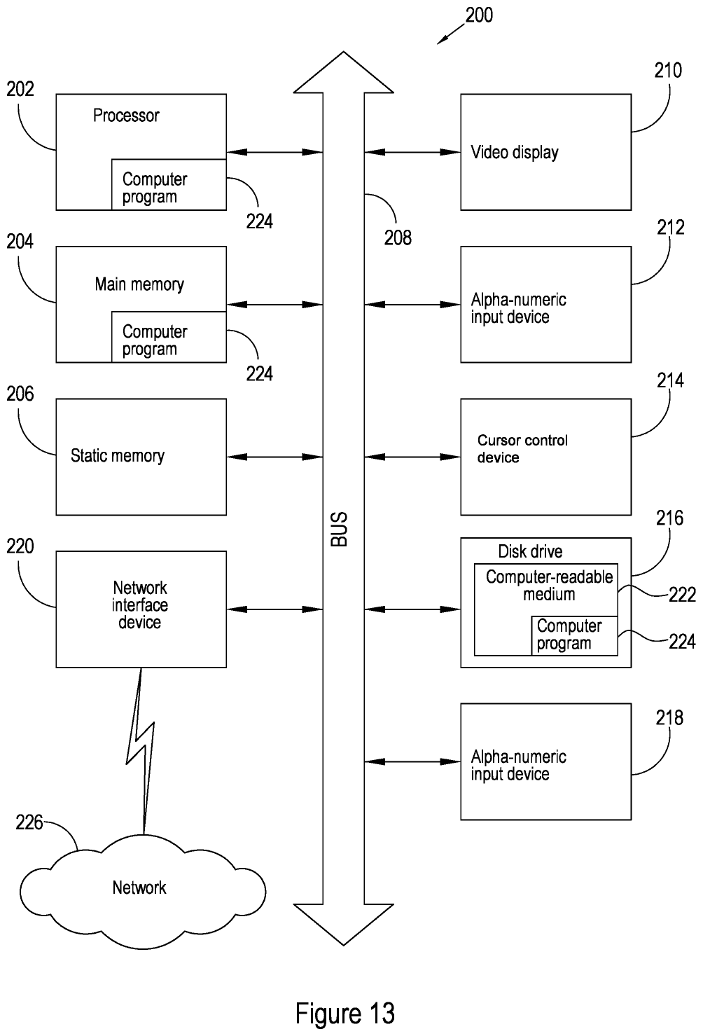

Referring now to FIG. 13 of the drawings which shows a diagrammatic representation of a machine in the example of a computer system 200 within which a set of instructions, for causing the machine to perform any one or more of the methodologies discussed herein, may be executed. In other example embodiments, the machine operates as a standalone device or may be connected (e.g., networked) to other machines. In a networked example embodiment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or ridge, or any machine capable of executing a set of instructions (sequential or otherwise) that specify actions to be taken by that machine. Further, while only a single machine is illustrated for convenience, the term “machine” shall also be taken to include any collection of machines, including virtual machines, that individually or jointly execute a set (or multiple sets) of instructions to perform any one or more of the methodologies discussed herein.

In any event, the example computer system 200 includes a processor 202 (e.g., a central processing unit (CPU), a graphics processing unit (GPU) or both), a main memory 204 and a static memory 206, which communicate with each other via a bus 208. The computer system 200 may further include a video display unit 210 (e.g., a liquid crystal display (LCD) or a cathode ray tube (CRT)). The computer system 200 also includes an alphanumeric input device 212 (e.g., a keyboard), a user interface (UI) navigation device 214 (e.g., a mouse, or touchpad), a disk drive unit 216, a signal generation device 218 (e.g., a speaker) and a network interface device 220.

The disk drive unit 216 includes a non-transitory machine-readable medium 222 storing one or more sets of instructions and data structures (e.g., software 222) embodying or utilized by any one or more of the methodologies or functions described herein. The software 222 may also reside, completely or at least partially, within the main memory 204 and/or within the processor 202 during execution thereof by the computer system 200, the main memory 204 and the processor 202 also constituting machine-readable media. The software 222 may further be transmitted or received over a network 226 via the network interface device 220 utilizing any one of a number of well-known transfer protocols (e.g., HTTP). Although the machine-readable medium 222 is shown in an example embodiment to be a single medium, the term “machine-readable medium” may refer to a single medium or multiple medium (e.g., a centralized or distributed memory store, and/or associated caches and servers) that store the one or more sets of instructions. The term “machine-readable medium” may also be taken to include any medium that is capable of storing, encoding or carrying a set of instructions for execution by the machine and that cause the machine to perform any one or more of the methodologies of the present invention, or that is capable of storing, encoding or carrying data structures utilized by or associated with such a set of instructions. The term “machine-readable medium” may accordingly be taken to include, but not be limited to, solid-state memories, optical and magnetic media, and carrier wave signals.

While the invention has been described in detail with respect to a specific embodiment and/or example thereof, it will be appreciated that those skilled in the art, upon attaining an understanding of the foregoing may readily conceive of alterations to, variations of and equivalents to these embodiments.

1. A ventilation monitoring system for use in monitoring ventilation of at least one patient being ventilated by a suitable ventilator device via an associated connection arrangement defining a flow path of gas including air or oxygen between the patient and the ventilator device, wherein the system comprises:
a ventilation data capturing device connected between a patient and a ventilator device dedicated to and/or associated with the patient, wherein the ventilation data capturing device is arranged to collect ventilation data from at least the flow path between the patient and the ventilator device, wherein the ventilator device and/or ventilation data capturing device comprise unique identifiers;
at least one remote processor; and
at least one memory device coupled to the at least one remote processor, wherein the at least one memory device containing instructions which, when executed by the at least one remote processor are arranged to cause the at least one remote processor to:
associate and store in the at least one memory device, unique identifiers of at least two of an associated ventilator device, ventilation data capturing device, and the patient under a particular ventilation profile or ventilation file;
collect or receive ventilation data from the ventilation data capturing device; and
generate output data, based on the collected or received ventilation data, wherein the output data comprises or is indicative of a status of the ventilator device, or
generate output data based on at least one of the collected or received ventilation data, status of the ventilator device determined from the received or collected ventilation data, and commands collected from a remote device that is in communication with at least the ventilation data capturing device, wherein the output data comprising at least instructions/commands for actioning on the ventilator device associated with the ventilation data capturing device to allow suitable ventilation to be provided by the ventilator device to the patient.

2. The ventilation monitoring system according to claim 1, wherein the ventilation data capturing device is connected in an in-line flow communicative fashion in the flow path between the ventilator device and the patient.

3. The ventilation monitoring system according to claim 1, wherein the ventilation data includes at least pressure data indicative of and/or associated with a ventilation process between the patient and the ventilator device, wherein the ventilation process includes the creation and/or generation of positive and/or negative pressure differentials between the airways and lungs of a patient to support and/or provide for inhalation and/or exhalation of the patient, and thus wherein the pressure data is indicative of gas pressure in the flow path between ventilator device and the patient.

4. The ventilation monitoring system according to claim 3, wherein the pressure data comprises pressure signals, or data indicative thereof, associated with the ventilation process, and wherein the ventilation data capturing device comprises one or more suitable sensors for sensing at least gas pressure associated with or indicative of the ventilation process and generating pressure signals in response to said sensing.

5. The ventilation monitoring system according to claim 1, wherein the ventilation data capturing device comprises a memory, a communication module and a controller, wherein the controller is configured to transmit ventilation data collected by the ventilation data capturing device via the one or more sensors to the at least one remote processor, via the communication module, and wherein the at least one remote processor is configured to:
analyse the ventilation data collected by the ventilation data capturing device; and
generate output data, based on the analysed ventilation data, wherein the output data comprises at least visual representations of the analysed and/or collected ventilation data.

6. The ventilation monitoring system according to claim 5, wherein the output data comprises diagrams and/or graphs indicative of the ventilation process including pressure versus time diagrams; flow versus time diagrams; and/or air volume versus time diagrams.

7. The ventilation monitoring system according to claim 6, wherein the analysis of the ventilation data includes performing process diagnostics on the ventilation data to extract set points of the ventilation process, wherein the set points are selected from a group comprising an inspiration:expiration ratio; peak inhalation pressure; exhalation pressure; and tidal volume.

8. The ventilation monitoring system according to claim 1, wherein the at least one remote processor is configured to collect or receive patient metadata including information indicative of one or more of the patient's gender, patient's age, ventilation device details, and details of the patient circuit tubing selection.

9. The ventilation monitoring system according to claim 8, wherein the at least one remote processor is further configured to:
analyse the patient metadata; and
generate output data, wherein the output data comprises at least visual representations of the analysed patient metadata.

10. The ventilation monitoring system according to claim 1, wherein the at least one remote processor is further configured to collect commands/instructions, from the remote device, for displaying on an on-site device associated with an on-site medical practitioner for actioning by the on-site medical practitioner on the ventilator device.

11. The ventilation monitoring system according to claim 1, wherein the at least one remote processor is arranged to generate an alarm when the collected ventilation data is below or above a predefined threshold, wherein the ventilation data includes information indicative of the gas pressure flowing from the ventilator device to the patient, wherein the gas pressure is indicative of whether the gas pressure is higher or lower than required for providing adequate ventilation to the patient.

12. The ventilation monitoring system according to claim 7, wherein the at least one remote processor is arranged to collect and store extracted, historic set points, and further wherein the at least one remote processor is arranged to compare, substantially in real-time, extracted historic set points of the patient with extracted, current set points, and when the extracted, current set points deviate from the extracted, historic set points of the patient, the at least one remote processor is arranged to generate a suitable alarm or alert message.

13. The ventilation monitoring system according to claim 1, wherein in generating an output comprising the status of the ventilator device, wherein the ventilator device comprises a gas cylinder and a gas mask connectable to the gas cylinder, the at least one remote processor is arranged to determine, substantially in real-time, an amount of gas remaining in the ventilator device based on a flow rate of gas travelling from the ventilator device to the patient over a period of time.

14. The ventilation monitoring system according to claim 13, wherein if the amount of gas remaining in the ventilator device falls below a predetermined threshold, the at least one remote processor is arranged to generate a suitable alarm or alert message.

15. The ventilation monitoring system according to claim 1, wherein in generating an output comprising the status of the ventilator device, the at least one remote processor is arranged to determine, substantially in real-time, an operating condition of the ventilator device by determining whether there is a flow of gas or a reduction of flow of gas from the ventilator device to the patient, wherein the absence of or reduction of a flow of gas between the ventilator device and the patient is indicative that the ventilation device is not functioning.

16. The ventilation monitoring system as claimed in claim 15, wherein in the absence of or reduction of a flow of gas between the ventilator device and the patient, the at least one remote processor is arranged to generate a suitable alarm or alert message.

17. A ventilation monitoring system according to claim 1 for use in a method of monitoring ventilation of at least one patient

18. A computer-readable medium storing instructions thereon which are executable by at least one remote processor of a ventilation monitoring system, the ventilation monitoring system comprising:
a ventilation data capturing device connected between a patient and a ventilator device dedicated to and/or associated with the patient, for providing ventilation support to the patient, the ventilation data capturing device being arranged to collect ventilation data from a gas flow path defined by a suitable connection arrangement between the patient and ventilator device, wherein the ventilator device and/or ventilation data capturing device comprise unique identifiers; wherein the instructions when executed by the at least one remote processor are arranged to cause the at least one remote processor to perform the operations of:
associating and storing in the computer readable-medium or at least one memory device, unique identifiers of at least two of an associated ventilator device, ventilation data capturing device, and the patient under a particular ventilation profile or ventilation file;
collecting ventilation data from the ventilation data capturing device; and
generating output data, the output data indicative of, or comprising, a status of the ventilator device, or
generating output data based on at least one of the status of the ventilator device determined from the collected ventilation data, received or collected ventilation data from the ventilation data capturing device, and commands collected from a remote device that is arranged to be in communication with at least the ventilation data capturing device, wherein the output data comprising at least instructions or commands for actioning on the ventilator device associated with the ventilation data capturing device to allow suitable ventilation to be provided by the ventilator device to the patient.

19. A ventilation monitoring system for use in monitoring ventilation of at least one patient being ventilated by a suitable ventilator device via an associated connection arrangement defining a flow path of gas including air or oxygen between the patient and the ventilator device, wherein the system comprises:
the ventilator device, wherein the ventilator device comprises a gas canister containing suitable gas for ventilation of the patient;
a ventilation data capturing device connected between the patient and the ventilator device dedicated to and/or associated with the patient, and wherein the ventilation data capturing device is arranged to collect ventilation data from at least the flow path between the patient and the ventilator device, and further wherein the ventilator device and/or ventilation data capturing device comprise unique identifiers;
at least one remote processor; and
at least one memory device coupled to the at least one remote processor, wherein the at least one memory device containing instructions which, when executed by the at least one remote processor are arranged to cause the at least one remote processor to:
associate and store in the at least one memory device, unique identifiers of at least two of an associated ventilator device, ventilation data capturing device, and the patient under a particular ventilation profile or ventilation file;
collect or receive ventilation data from the ventilation data capturing device; and
generate output data based on the collected or received ventilation data, wherein the output data comprises or is indicative of a status of the ventilator device, and wherein in generating the output comprising the status of the ventilator device, the at least one remote processor is further arranged to determine, substantially in real-time, the amount of gas remaining in the ventilator device based on a flow rate of gas travelling from the ventilator device to the patient over a period of time.
